(19) United States
(12) Patent Application Publication
(10) Pub. No.: US 2005/0042654 A1
(43) Pub. Date: Feb. 24, 2005

(54) Genotyping methods

(75) Inventors: Rui Mei, Santa Clara, CA (US); Patrick Sean Walsh, Danville, CA (US); Shoulian Dong, San Jose, CA (US); Pat Tanti, Campbell, CA (US); William A. Lyon, Sacramento, CA (US); Gretchen L. Schieber, San Jose, CA (US); Mark Kelleher, San Jose, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/880,143

(22) Filed: Jun. 28, 2004

Related U.S. Application Data
(60) Provisional application No. 60/556,753, filed on Mar. 26, 2004.
(60) Provisional application No. 60/483,050, filed on Jun. 27, 2003.

(51) Int. Cl. C12Q 1/68; C12P 19/34; C12M 1/34

(52) U.S. Cl. 435/6; 435/91.2; 435/287.2

(57) ABSTRACT

Methods for amplifying genomic DNA and genotyping amplified genomic DNA samples are provided. The genotyping methods use genotyping arrays of probes that are allele specific probes for single nucleotide polymorphisms (SNPs). The methods also relate to methods for amplifying a plurality of genomic DNA samples from a plurality of individuals in a manner that minimizes the potential for contamination of samples that have not been amplified by amplicons from samples that have already been amplified.

### RELATED APPLICATIONS

The present application claims priority to U.S. Provisional Patent Application Nos. 60/483,050 filed Jun. 27, 2003 and 60/556,753 filed Mar. 26, 2004, the disclosures of which are incorporated herein by reference in their entireties.

### FIELD OF THE INVENTION

The invention relates to methods of genotyping large numbers of single nucleotide polymorphisms (SNPs) in parallel by hybridization of amplified sample to arrays of allele specific oligonucleotide probes. Methods for high throughput genotyping analysis are provided. In some embodiments methods, kits and systems for minimizing cross contamination in a genotyping system are provided. The present invention relates to the fields of molecular biology and genetics.

### BACKGROUND OF THE INVENTION

The past years have seen a dynamic change in the ability of science to comprehend vast amounts of data. Pioneering technologies such as nucleic acid arrays allow scientists to delve into the world of genetics in far greater detail than ever before. Exploration of genomic DNA has long been a dream of the scientific community. Held within the complex structures of genomic DNA lies the potential to identify, diagnose, or treat diseases like cancer, Alzheimer disease or alcoholism. Exploitation of genomic information from plants and animals may also provide answers to the world's food distribution problems.

Recent efforts in the scientific community, such as the publication of the draft sequence of the human genome in February 2001, have changed the dream of genome exploration into a reality. Genome-wide assays, however, must contend with the complexity of genomes; the human genome for example is estimated to have a complexity of 3×10⁹ base pairs. Novel methods of sample preparation and sample analysis that reduce complexity may provide for the fast and cost effective exploration of complex samples of nucleic acids, particularly genomic DNA.

### SUMMARY OF THE INVENTION

In one embodiment a system for high throughput detection of genotypes of a plurality of single nucleotide polymorphisms (SNPs) in a plurality of genomic samples is disclosed. The system comprises a sample preparation method that includes an amplification step, a fragmentation step; and a labeling step to label the fragments. This generates genomic DNA amplicons that are fragmented and labeled and ready for hybridization to a genotyping array. A sample preparation kit that includes a first and second container and optionally a third container is used for the preparation of the sample. One container contains reagent stocks for amplification of genomic samples and the second container contains reagent stocks for fragmentation and labeling of amplified genomic samples. The system further comprises areas including a first low copy lab area wherein the components of the first container are stored and wherein reagent mixtures comprising the components of the first container are aliquoted into reagent master mixtures in preparation for assembly of amplification reactions; a second low copy area where the plurality of genomic DNA samples is stored and wherein a plurality of amplification reactions is assembled, wherein each amplification reaction comprises an aliquot of a reagent master mixture and an aliquot of a genomic DNA sample from the plurality of genomic DNA samples; and a high copy lab area where amplification reactions are incubated under amplification conditions to generate a plurality of amplified genomic DNA samples. The system also comprises instructions to lab personnel to restrict movement of amplified genomic DNA samples, lab personnel and equipment from the high copy lab area to said first and second low copy lab areas. After sample preparation the prepared sample is hybridized to a plurality of genotyping arrays.

In preferred embodiments genotyping arras comprise a set of at least 200,000 probes comprising at least 10,000 probe sets wherein a probe set comprises at least 20 probes that are each complementary to a 20 to 30 base region comprising a single nucleotide polymorphism and the probe set comprises probes that are perfectly complementary to a first allele of the SNP and probes that are perfectly complementary to a second allele of the SNP. A hybridization pattern is generated for each genomic DNA sample and a computer system is used to analyze the hybridization pattern to make genotype calls for a plurality of SNPs that are interrogated by the array.

In a preferred embodiment the computer system comprises a processor; and a memory that is coupled with the processor, the memory storing a plurality of machine instructions that cause the processor to perform the method step of analyzing the hybridization to determine the genotype. The system may further comprises a sample tracking system that may be a bar code system or an electromagnetic encoding system. In preferred embodiments one or more steps are automated, for example a robot that handles multiwell plates may be used for sample preparation.

The low and high copy areas preferably contain separate copies of equipment such as thermal cyclers, repeating pipettes and multi channel pipettes so that the equipment is not moved from area to area.

In another embodiment a method of determining the genotype of a plurality of SNPs in a genomic DNA sample is disclosed. A plurality of amplification reagent stocks are stored in a first low copy lab area. Genomic DNA samples and amplicons, for example PCR amplicons, are not brought into said first low copy lab area to minimize the chance for contamination of reagent stocks with genomic DNA or amplified DNA fragments. An amplification reagent master mix is assembled in the first low copy lab area. The amplification reagent master mix comprises aliquots of amplification reagent stocks. The amplification reagent master mix is transported to a second low copy lab area where unamplified genomic DNA samples are stored. Amplification reactions are stored in the second low copy lab area. The amplification reaction comprises an aliquot of unamplified genomic DNA and an aliquot of the amplification reagent master mix. The amplification reaction is transported to a high copy area, where reagent stocks for fragmentation and labeling of amplified samples are stored. The performing amplification reaction, fragmentation of the amplified sample and labeling of the fragments are performed in the high copy area. Labeled fragments are hybridized to a genotyping array and the hybridization pattern is analyzed to determining the genotype of a plurality of SNPs.

In one embodiment a kit for amplifying a genomic DNA sample is disclosed. The kit may include three separate containers each containing different reagents to be stored in different areas. The containers may be contained within a larger container. In a preferred embodiment each of the inner containers is labeled according to where the container or the reagents in the container should be stored, for example, in the first low copy lab area, the second low copy lab area or the high copy lab area. The first container may contain an adaptor and an amplification primer and optionally a ligase, a ligase buffer, a DNA polymerase, and a buffer for the DNA polymerase. The second container may contain a reference genomic DNA sample that is of known quality and known genotype. The third container may contain a DNase, a DNase buffer, a terminal deoxynucleotidyl transferase, a terminal deoxynucleotidyl transferase buffer, and a labeled nucleotide, for example, a biotinylated nucleotide such as GeneChip DNA Labeling Reagent, available from Affymetrix. The kit may also contain instructions for storing the contents of the different containers in different areas, according to where they will be used. The amplification primer may be, for example, a universal or common primer, random primers or degenerate or partially degenerate primers.

In one embodiment a panel of more than 10,000 SNPs may be genotyped in at least 96 individuals in parallel. A genomic DNA sample is isolated from each of the individuals and an aliquot of the sample is fragmented. An adaptor is ligated to the fragments in each fragmented genomic DNA sample to generate an adaptor-ligated genomic DNA sample for each individual. The first fragmentation and adaptor ligation steps are performed in a first low copy lab area. An amplification reaction is assembled in the low copy lab area for each individual by mixing an aliquot of adaptor ligated genomic DNA and reagents for amplification. The amplification reactions are incubated under amplification conditions to generate amplicons in a high copy lab area. Amplified samples from the high copy lab area are not intentionally transported back to the first low copy lab area after amplification.

The amplicons are fragmented, end labeled with a detectable label and hybridized to a genotyping array to generate a hybridization pattern for each genomic DNA sample. The hybridization pattern for each genomic DNA sample is analyzed using a computer system to determine the genotype of a plurality of SNPs in each of the individuals. The genotyping array may contain allele specific probes for each allele of more than 10,000, more than 100,000 or more than 1,000,000 SNPs from humans or from another mammal such as mouse or rat.

### BRIEF DESCRIPTION OF THE FIGURES

FIG. 1 shows a schematic of a method of amplifying a reduced complexity genomic sample, fragmenting and labeling the sample and hybridization of the sample to an array of probes.

FIG. 2 shows a schematic of a genotyping reagent kit box. The exterior box (101) contains three smaller boxes. Box 1 (103) is stored in a room free of DNA templates. Box 2 (107) is stored in a room free of amplicons. Box 3 (109) may be stored in a room where amplicons are present. In a preferred embodiment, box 1 is stored in a Pre-PCR Clean Area, box 2 in a PCR staging area and box 3 in the area where PCR amplification takes place.

### DETAILED DESCRIPTION OF THE INVENTION

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/13,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication Number 20020183936), 10/065,856, 10/065,868, 10/328,818, 10/328,872, 10/423,403, and 60/482,389.

b) Definitions

The term “admixture” refers to the phenomenon of gene flow between populations resulting from migration. Admixture can create linkage disequilibrium (LD).

The term “allele” as used herein is any one of a number of alternative forms a given locus (position) on a chromosome. An allele may be used to indicate one form of a polymorphism, for example, a biallelic single nucleotide polymorphism (SNP) may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, the base or bases present at a single SNP. For example, if a SNP is biallelic and can be either an A or a C then if an individual is homozygous for A at that position the genotype of the SNP is homozygous A or AA. Genotype may also refer to the information present at a plurality of polymorphic positions.

Genotyping arrays and methods are described, for example, in U.S. patent application Ser. Nos. 10/681,773, 10/316,517, 10/442,021, 10/740,230, 60/495,606 and 09/904,039, the disclosures of which are incorporated herein by reference in their entireties. In preferred embodiments genotyping arrays comprise a set of probes for each of a pre-selected set of known SNPs. The probe sets may comprise 10 to 80 probes, preferably comprising about 40 probes. Some of the probes in the probe set are perfectly complementary to a first allele of a SNP and other probes in the probe set are perfectly complementary to a second allele of a SNP. The genotype of the SNP in the sample is determined by allele specific probe hybridization. Some of the probes in the probe set are mismatch control probes. The position of the SNP base is preferably shifted relative to the center of the probe in different probes in a probe set.

The term “Hardy-Weinberg equilibrium” (HWE) as used herein refers to the principle that an allele that when homozygous leads to a disorder that prevents the individual from reproducing does not disappear from the population but remains present in a population in the undetectable heterozygous state at a constant allele frequency.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage analysis” as used herein refers to a method of genetic analysis in which data are collected from affected families, and regions of the genome are identified that co-segregated with the disease in many independent families or over many generations of an extended pedigree. A disease locus may be identified because it lies in a region of the genome that is shared by all affected members of a pedigree.

The term “linkage disequilibrium” or sometimes referred to as “allelic association” as used herein refers to the preferential association of a particular allele or genetic marker with a specific allele, or genetic marker at a nearby chromosomal location more frequently than expected by chance for any particular allele frequency in the population. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. The genetic interval around a disease locus may be narrowed by detecting disequilibrium between nearby markers and the disease locus. For additional information on linkage disequilibrium see Ardlie et al., Nat. Rev. Gen. 3:299-309, 2002.

The term “lod score” or “LOD” is the log of the odds ratio of the probability of the data occurring under the specific hypothesis relative to the null hypothesis. LOD=log [probability assuming linkage/probability assuming no linkage].

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population, but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms. Many human SNPs have been identified, see, for example, The International SNP Map Working Group, Nature 409, 928-33 (2001) and Wang, D. G., et al., Science 280, 1077-1082 (1998).

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Parallel Genotyping of Multiple SNPs in Multiple Samples

The millions of SNPs that have been identified in the human genome (see, for example, Sachidanandam, et al. 2001) provide a large repository of markers for human variation, allowing construction of increasingly dense SNP maps and tools for analysis of large numbers of individual SNP markers. These tools have the potential of enabling investigators to generate high resolution mapping of complex human genetic traits, to understand the history of human populations, and to examine genomic abnormalities, such as chromosomal copy number changes that lead to cancer and other diseases.

Recent estimates suggest that there may be ˜5 million SNPs with minor allele frequencies of at least 10%, and possibly as many as ˜11 million with minor allele frequencies of at least 1% (Kruglyak, L. and D. A. Nickerson. 2001. Nat Genet 27: 234-236). Millions of human SNPs have been catalogued, many of which are publicly available in the TSC and NCBI dbSNP repositories (Thorisson, G. A. and L. D. Stein. 2003. Nucleic Acids Res 31: 124-127). Alternative non-locus specific approaches, such as degenerate oligonucleotide primer (DOP)-PCR, that genotype SNPs in a reduced complexity fraction of the genome may be used (Jordan, et al. 2002. Proc Natl Acad Sci USA 99: 2942-2947; Grant, et al. 2002. Nucleic Acids Res 30: e125). High density oligonucleotide arrays have been used to investigate polymorphisms (Chee, et al., 1996. Science 274: 610-614.), and have been applied to SNP genotyping (Carrasquillo et al. 2002 Nat Genet 32: 237-244; Fan et al 2002 Genome Res 10: 853-860; Fan et al 2000 Genomics 79: 58-62; Hacia et al 1999 Nat Genet 22: 164-167; Wang et al 1998 Science 280: 1077-1082).

Assays that provide methods for genotyping of large numbers of SNPs may be used for linkage and association studies. Additionally, a higher density of SNPs increases the proportion of linkage disequilibrium (LD) measured in the sample. High density SNP genotyping assays may also be used to map and measure blocks of LD in isolated populations. Dense sets of SNP markers may be used to capture LD for mapping complex diseases and to perform studies of cancer where there is chromosomal instability, denser sets of markers may be used for finer resolution in determining the borders of chromosomal losses and gains.

Methods are provided for genotyping more than 10,000, 100,000 or 1,000,000 SNPs in parallel from a genomic sample. Methods are also provided for two laboratory personnel to simultaneously genotype the same set of more than 10,000 or more than 100,000 SNPs from more than 95, 200, 500, 700, or 1400 samples simultaneously without the use of robots or automation, although robots and automation may be used in some embodiments. A highly reproducible generic sample preparation method is combined with allele-specific hybridization as described in Kennedy, et al. Nat. Biotechnol 21, 1233-7 (2003).

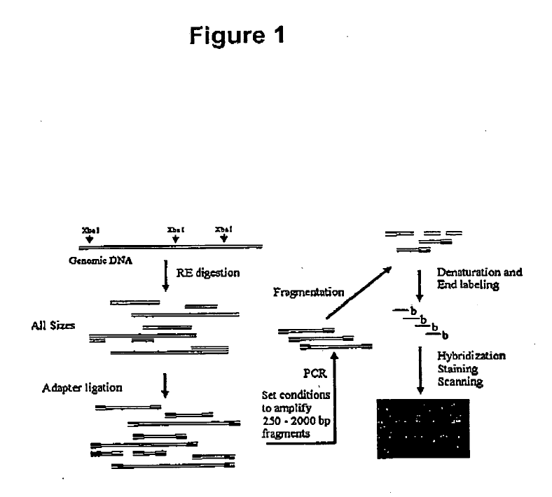

A schematic of an amplification method useful for one embodiment of the method is shown in FIG. 1. Sample genomic DNAs are digested with a restriction enzyme such as Xba I; then adaptors are ligated to the ends of restriction fragments, and the fragments are amplified using one of the strands of the adaptor as a primer. Restriction fragments in the size range, 250 to 2000 base pairs (bp), are preferentially amplified most efficiently while fragments smaller than 250 bp and greater than 2000 bp are less efficiently amplified. The narrow size range of amplicons (amplified fragments) is estimated to represent approximately 60 Mb of sequence complexity, which is approximately a 50-fold reduction in the complexity of the human genome. The PCR products are fragmented with DNAse I. The size range of the fragmented PCR products is shown in the second gel image. The fragmented products are biotinylated and then hybridized to the arrays. Following a series of stringent washing and signal generation steps, the arrays are scanned; and genotypes are determined based on hybridization signal intensities.

In a preferred embodiment an aliquot of genomic DNA from a sample is fragmented, amplified and analyzed in two parallel reactions, each using a different restriction enzyme and each being hybridized to a different array of probes. Each restriction enzyme generates a different pool of fragments that are between 250 and 2000 base pairs so different SNPs will be present in the amplified sample depending on which enzyme was used for digestion. Allele specific probe sets are used to interrogate the genotypes of each SNP so the amplified fractions are hybridized to arrays of probes that are specific for the SNPs that are present in that amplified fraction. For example, if Xba I is used for digestion SNPs that are on 250 to 2000 bp XbaI fragments, the amplified sample will be hybridized to an array of probes that interrogate SNPs in that fraction which will be different from the SNPs present in the fraction that results if the sample is fragmented with HindIII. A separate array will be used to interrogate the HindIII fraction. A single primer is used for amplification. The primer is complementary to the adaptor sequence that is ligated to the fragments. The adaptors may be similar with changes as necessary in the overhanging region depending on the overhang left by the enzyme used for digestion. The method is highly reproducible in that essentially the same fragments may be amplified reproducibly across many samples. This method may be used in a single primer assay to genotype more than 10,000, 100,000, 500,000 or 1,000,000 SNPs by allele specific hybridization to an array of probes.

The methods may be used to identify regions of the genome linked to or associated with a particular trait or phenotype. Additionally, methods useful for determination of allele frequencies in various populations and for mapping regions with loss of heterozygosity during cancer progression are disclosed. Genotyping arrays have been used for LOH analysis, see, for example, Lindblad-Toh, K., et al., Nat. Biotechnol 18, 1001-1005 (2000).

In one embodiment a computer is used to predict the fragments that will result when a selected reference genome is fragmented with a selected restriction enzyme, this is referred to as in silico fractionation. The predicted fragments are then used to design probes for an array. For example, each fragment that is within a selected size range that contains a previously identified SNP may be the target of a genotyping probe set on an array. A sample that contains genomic DNA that is from the same species as the reference genome or a related species is then fractionated biochemically according to the disclosed methods or other methods that result in predictable, reproducible complexity reduction and amplification. The reduced complexity amplified sample may then be fragmented and labeled and hybridized to an array of allele-specific probes for genotyping the SNPs in the fragments.

Methods of allele specific hybridization using high density DNA arrays to determine genetic information have been described, for example, in Chee et al. Science 274, 610-614 (1996), Matsuzaki et al., Genome Res 14: 414-25, (2004) and Liu et al., Bioinformatics 19:2397-403, (2003). See also, U.S. patent application Ser. No. 10/740,230.

In a preferred embodiment the amplification reaction is preformed using a work flow system and laboratory set up that minimizes the opportunity for cross contamination of samples. Preferably the work flow proceeds in a single direction so that amplified products and those items that may come in contact with amplified products are not brought into the low copy areas where unamplified samples and reagents for amplification and for steps prior to amplification are stored and accessed.

Methods of analysis of genomic DNA that involve an amplification step that uses generic, non locus-specific, primers are susceptible to contamination of unamplified samples by amplicons from other samples. The contaminating amplicons will be amplified during the subsequent amplification of the contaminated sample and may interfere with the analysis of the sample. Generic amplification methods, for example, the single primer amplification shown in FIG. 1, other amplification methods that attach universal priming sequences to genomic fragments then amplify using universal primers (for example, US Patent Pub. 20030143599) and amplification methods that use random primer sequences, for example MDA (for example, US Patent Pub. 20030143587), are sensitive to this type of contamination and the disclosed methods, systems and kits may be useful for any of these amplification methods.

For methods that use generic primer amplification methods a likely potential source of contamination is previously amplified product. In a preferred embodiment steps are taken to minimize the possibility of contaminating pre-amplification steps with amplified product. In a preferred embodiment individual steps in the preparation of genomic samples for hybridization to arrays are performed in distinct areas. Preferably the methods separate reagents, equipment and consumables that may come into contact with unamplified samples from reagents, equipment and consumables that may come into contact with amplified samples. In this manner the reagents, equipment and consumables that may contain contaminating amplicons do not come into contact with the unamplified samples, or the reagents, equipment and consumables that may come into contact with unamplified samples. Steps may also be taken to prevent contamination of areas that are identified as low copy areas by amplicons that are carried on the person or clothing of lab personnel. In a preferred embodiment amplification is by PCR or multiple displacement amplification.

In one embodiment the reagents that are used for amplification are present in a kit that is separable into sub-kits that are used in specified areas and are preferably stored in the areas where they will be used. The kit includes instructions, which may be labels on one or more containers that instruct the lab personnel to store individual reagent components of the kit in specified areas. Although many of the examples provided specify the amplification method as being by PCR this is simply illustrative of the methods and the methods are not limited to PCR amplification methods. The methods are useful for any amplification method and particularly for methods that employ priming with primers that are universal, degenerate, random, generic or not locus specific. For additional information about methods of amplification that utilize non locus-specific amplification see, for example, U.S. Pat. No. 6,703,228 which is incorporated herein by reference in its entirety. Non locus-specific amplification methods use a limited number of primers to amplify many sequences from a genome.

In one embodiment all of the reagents necessary for preparation of the PCR reagents are provided in a first box, all the reagents necessary for PCR staging, including the steps of enzyme digestion, libation and set up of the PCRs are provided in a second box, and all the reagents necessary for PCR thermal cycling, fragmentation and labeling are provided in a third box.

In one embodiment the first box may include the adaptor and the PCR primer. The second box may include any of the following: a control genomic DNA, PCR buffer, a thermal stable polymerase, dNTPs, one or more restriction enzymes, buffer for restriction enzymes, and ligase. The third box may have any of the following: fragmentation reagent, fragmentation buffer, labeling reagent, terminal deoxynucleotidyl transferase TdT, TdT buffer, oligonucleotide control reagent, and SAPE.

In many embodiments each box is stored in the room in which it will be used and the boxes are not stored together after initial unpacking. In one embodiment the three boxes are shipped together in one larger box and separated before use.

A universal primer as used herein is directed to a single sequence that is added to a plurality of different sequences. The universal priming site that is added is preferably not naturally occurring in the genomic DNA sample.

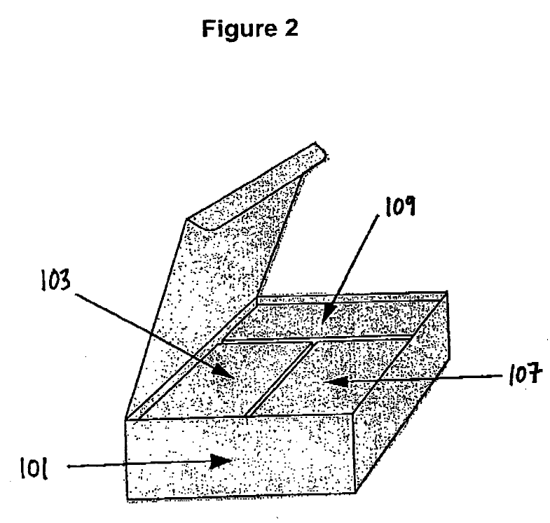

One preferred embodiment of a kit that is useful for the methods is shown in FIG. 2 which shows a schematic of a container for a kit comprising some of the reagents used to perform the amplification method shown in FIG. 1. The outer container (101) is provided as a means of initially delivering and storing the kit. When the kit is opened by the lab personnel the inner containers, (103, 107 and 109) are labeled according to the area where they should be stored and used. The first box (103) contains the adaptor for the adaptor ligation step and the PCR primer and may be stored in the “Pre-PCR Clean Area”, the second box (107) contains a control genomic DNA sample to be used as a reference or for trouble shooting the method and may be stored in the “PCR Staging Area”, box 3 (109) contains 10×fragmentation buffer, fragmentation reagent, TdT, TdT buffer, GeneChip DNA labeling reagent and control oligonucleotide B2 and may be stored in the “Main Lab” which is a high copy lab area.

The Pre-PCR Clean Area is a low copy lab area and is preferably maintained so that it is substantially free of sample DNA templates and amplified products, steps to minimize the amount of DNA template that may be present in this area may include, instructing lab personnel to not bring genomic DNA samples, amplified samples or anything that may be contaminated with genomic DNA samples or amplified samples into the area intentionally and cleaning the area, for example, the lab bench surfaces and equipment in the area to remove DNA that may be present. The areas may be cleaned, for example, with a bleach solution. In a preferred embodiment reagent stocks are prepared and stored in the Pre-PCR Clean Area, this may include removing aliquots of reagent for an experiment from reagent stocks to set up master mixes to be aliquoted into individual samples. Particular care is taken with reagent stocks because they are used multiple times and may be aliquoted into many different genomic samples. Contamination of a reagent stock may result in wide spread contamination of amplification reactions. Reagent stocks may be, for example, 10×buffer solutions, undiluted enzymes, adapter solutions, and concentrated solutions of dNTPs.

The PCR Staging Area is also a low copy lab area and is preferably maintained so that it is substantially free of amplified product. The genomic DNA samples are brought into this area and may be stored here prior to amplification. This area is where master mixes of reagents may be added to individual reactions and some reactions such as fragmentation and ligation may be performed in this area. For the method shown in FIG. 1 the enzyme digestion and ligation steps are performed in the PCR Staging Area and PCRs for genomic samples are set up in this area, for example, master mixes of the PCR reagents are added to each sample. In a preferred embodiment the genomic samples are present in multiwell plates, such as 96 or 384 well microtitre plates for many of the steps of the method. Genomic DNA fragmentation, adaptor ligation, PCR, fragmentation of PCR amplicons, denaturation and labeling may all be performed in multiwell plates. In a preferred embodiment lab personnel wear protective clothing that prevents dust, human skin and hair particles from entering the room's atmosphere, similar to the clothing worn in clean rooms. Attire may include a covering for the head and a mask to cover the mouth area. The use of gowns and gloves is recommended to prevent PCR carryover and will minimize the risk of trace levels of contaminants being brought into the Pre-PCR Clean Area. It is the area where non-amplified template (genomic DNA) should be handled. The use of gowns and gloves is recommended to prevent PCR carryover.

The Main Lab is a location where amplification takes place so by necessity amplicons are present in this area. This area preferably contains one or more thermal cyclers to be used for amplification. For the method shown in FIG. 1, for example, PCR thermal cycling, PCR clean up, fragmentation of the PCR, labeling, and hybridization, washing and staining of arrays may be done in the Main Lab. The procedures for the Main Lab are preferably designed to minimize movement of possible contaminating DNA amplicons from the Main Lab to the Pre-PCR Clean Room or the PCR Staging Area. These procedures may include, for example, instructing lab personnel not to enter the Pre-PCR Clean Room or the PCR Staging Room after being in the Main Lab unless they have taken steps to remove contaminating amplicons from their clothing and their person by, for example, showering and changing into freshly laundered clothing or protective lab attire; maintaining separate copies of any protocols or manuals in each of the areas where they may be referred to so that protocols are not moved from one area to the next; and having equipment that is dedicated to the areas so that equipment, such as pipettes, are not moved from one area to another.

In one embodiment reagents are provided in a kit that comprises three separate containers and each container is labeled to indicate the area where the container, or the reagents in the container, should be stored. In general the areas are pre amplification, amplification staging and amplification areas. Preferably personnel movement from the amplification areas to the pre amplification and amplification staging areas is restricted. Preferably dedicated equipment (e.g., pipettes etc.) is used for pre-amplification and amplification preparation stages and equipment is stored in the area where it is used.

The pre-amplification and amplification staging areas may also be referred to as “low copy lab areas” and the amplification area may be referred to as a “high copy lab area”. The areas may be separate rooms, or a dedicated area such as a separate lab bench or a hood, for example. One or more of the areas may be areas of restricted air flow such as a clean room or a laminar flow hood. Once an individual has entered a high copy area, that individual should not enter a low copy area until they have showered or changed into freshly laundered clothing or both.

Genomic DNA samples for processing may be isolated from any suitable source. Suitable sources include, for example, blood samples, cell lines, and buccal samples. Preferably samples are free of contaminating DNA from, for examples, microbes. Methods of isolating DNA from buccal samples are disclosed, for example, in Feigelson, et al., Cancer Epidemiol Biomarkers Prev. 10(9), 1005-8 (2001). Heath, et al., Arch Pathol Lab Med 125, 127-133 (2001). King, et al., Cancer Epidemiol Biomarkers Prev. 11(10 Pt 1), 1130-3 (2002) and Lench, et al., Lancet, 1(8599), 1356-1358 (1988).

In preferred embodiments genomic DNA is extracted and purified by a method that does not include boiling or strong denaturants that would render the DNA single-stranded. Methods that may be used are, for example, (1) SDS/proteinaseK digestion, phenol-chloroform extraction, Microcon or Centricon (Millipore) ultrapurification and concentration or (2) QIAamp DNA Blood Maxi Kit from Qiagen. In preferred embodiments a kit comprising a genomic DNA control (for example, Reference Genomic DNA, 103) may be provided along with other amplification reagents as a process control. Preferably the control DNA is of known genotype for the SNPs being assayed and may be used as a routine experimental positive control and/or for troubleshooting, as needed.

In a preferred embodiment genomic DNA samples to be processed are double-stranded to facilitate restriction enzyme digestion. DNA is preferably free of PCR or amplification inhibitors. Examples of inhibitors include high concentrations of heme (from blood) and high concentrations of chelating agents. To reduce the amount of inhibitors in a genomic DNA sample the sample may be subjected to a cleanup step prior to amplification. In one embodiment the following method may be used: add 0.5 volumes of 7.5 M NH₄OAc, 2.5 volumes of absolute ethanol (stored at −20° C.), and 0.5 μl of glycogen (5 mg/mL) to 250 ng genomic DNA; vortex and incubate at −20° C. for 1 hour; centrifuge at 12,000×g in a microcentrifuge at room temperature for 20 minutes, remove the supernatant and wash the pellet with 0.5 mL of 80% ethanol; centrifuge at 12,000×g at room temperature for 5 minutes; remove the 80% ethanol and repeat the 80% ethanol wash one more time; and re-suspend the pellet in TE (10 mM, pH 8.0, 0.1 mM EDTA, pH 8.0). This cleanup method may be used, for example, when samples are obtained from formalin fixed paraffin embedded tissue samples.

The genomic DNA extraction/purification method in a preferred embodiment results in DNA that has a low salt concentration or is substantially free of salt because high concentrations of certain salts can also inhibit PCR and other enzyme reactions. Preferably the DNA samples are not contaminated with other human genomic DNA sources, or with genomic DNA from other organisms.

In one embodiment the assay is optimized for 250 ng of input genomic DNA per restriction enzyme (for the first step, restriction enzyme digestion), at a nominal concentration of 50 ng/μL, but concentrations as low as approximately 17 ng/μL (250 ng/15.5 μL) may also be used. Genomic DNA of lower concentration can be concentrated, for example, using a Microcon—100 ultrafiltration unit.

Success with other types of samples such as formalin fixed paraffin-embedded tissue may depend on the quality (degree of degradation, degree of inhibitors present, etc.) and quantity of genomic DNA extracted, and on the purity of the sample. In a preferred embodiment samples that may contain inhibitors are subjected to a clean up step prior to amplification. The cleanup step preferably removes at least some of the inhibitor or inhibitors that are present and results in improved genotyping. The inhibitors may, for example, inhibit digestion, ligation or amplification.

In some embodiments the genomic DNA sample is subjected to an amplification step prior to the amplification step shown in FIG. 1. Methods of amplification that may be used include, for example, amplification with a phi29 enzyme as described in Paez et al. Nucleic Acids Res. 32(9):e71, (2004).

The one primer amplification method as shown in FIG. 1 may be used to reduce the complexity of the genome, and enables robust and efficient allele specific hybridization. In one embodiment the method involves five primary steps, starting with restriction digestion, ligation of adaptor, amplification, fragmentation, and labeling, that prepare samples for hybridization to the oligonucleotide array. The complexity reduction occurs at the PCR step which preferentially amplifies restriction fragments that are between 250 to 2000 bp. The adaptor sequence may be artificially generated to have no homology with known genome sequences. The one primer used in the PCR may be the sense strand of the adaptor; thus only two oligonucleotides are necessary for genotyping over 50,000 SNPs. Since the adaptor and primer sequence is not locus specific, the sequence may be interchanged with alternative sequences with no loss in PCR yields or changes in amplicon size distribution. Because the adaptor sequence is non-specific it may be changed to prevent amplification of carry over contamination. The primer and adaptor may be changed, for example after a time interval or after contamination is detected.

For the amplification method shown in FIG. 1, the sequence content of the reduced fraction of the genome is determined by the choice of restriction enzyme and PCR conditions. Each restriction enzyme defines a different fraction of the genome determined by the different locations of restriction sites in the genome, and the resulting sequence complexity is directly proportional to the frequency of restriction sites in the genome. Individual reaction conditions may vary within an optimal range without affecting call rates or concordance results significantly. See, for example, Affymetrix Technical Note, Optimization and Validation of the GeneChip Mapping Assay, 2003, Part No. 701368 Rev. 2.

The genomic DNA, amplified genomic DNA or PCR amplified target in one embodiment is fragmented prior to labeling and hybridization to an array. In one embodiment the DNA is fragmented with DNaseI or a similar nuclease. DNaseI gives a random distribution of fragments and the assay may be optimized to take advantage of this. The buffer conditions may be optimized to maximize call rate and concordance. A variety of conditions were tested to optimize concordance and call rate for the XbaI digest and the collection of approximately 11,500 SNPs predicted to be in the XbaI 200 to 1000 bp fraction. Buffer conditions and concentration of DNaseI were changed. The initial buffer system used functioned well at about 0.2 units of DNase. The optimized buffer system has a much wider range of DNase concentrations that give a call rate of about 95%. The optimized buffer therefore allows much greater room for error in DNase concentration that may result from changes in stability and activity.

DNA fragment size distribution is sensitive to the DNase amount used in each reaction. When a lower concentration of DNase is used, less digestion occurrs, resulting in longer fragments. Conversely, a higher concentration of DNase may be used to generate smaller fragment size. Different incubation time periods may also be used, 30 minutes of incubation is used in a preferred embodiment. Incubation periods from 25 to 35 minutes may be used.

In one aspect of the invention, a system for high throughput determination of genotypes is provided. The exemplary system includes a sample preparation method, a sample preparation automation system; a sample tracking system; an automated high density probe array loader; and a computer system for managing hybridization data and for analyzing hybridization data to make genotype calls.

In one embodiment a master mix of reagents is aliquoted into microwell strips, for example 8 or 12 well strips, and then dispensed into a multi well plate, for example a 96 well plate, with an 8 or 12 channel pipette. The mixture may be mixed by pipeting up and down several times. The multiwell plate may be covered with a plate cover that seals tightly and vortexed and briefly spun. The entire plate may then be placed in a thermal cycler for incubation at the appropriate temperature or for the appropriate number of cylces of a specified time at each of a series of temperatures.

The sample preparation automation system typically involves a robotic device for handling multiwell plates such as 96 well microtiter plates. In some embodiments, the sample tracking is performed using a machine readable encoding system, for example, a single dimensional or multiple dimensional barcode system or an electromagnetic encoding system. Suitable autoloaders are also described in, for example, U.S. patent application Ser. Nos. 09/691,702 and 60/396,457, which are incorporated herein by reference.

An autoloader provides a mechanism for transporting cartridges to and from a scanner. Conveniently, the invention may utilize standardized carriers that hold a number of cartridges that may be stored in a cool chamber. A two-axis robot may be employed to move the cartridges to and from the scanner, a warming station, and a holding station. A local operator interface and network connection may be provided to a host work station to facilitate operation of the transport system.

Use of the cartridge carriers is advantageous in that they provide a standardized way to hold the multiple cartridges. Further, the cartridge carriers may include keyed slots to prevent reverse installation. Use of the housing having a chilled chamber permits storage of the cartridges for several hours prior to scanning. However, it will be appreciated that in some embodiments, a temperature controlled chamber may not be needed. Following removal, the warming station may be used to eliminate condensation on the cartridge before its insertion into the scanner. Also, use of the robot allows automated movement of the cartridges between the carriers and the various stations in the scanner. Those of ordinary skill in the art will appreciate that many possible methods and components exist for the storage and automatic transport of probe array cartridges.

Additional examples of autoloaders are described in U.S. Provisional Patent Application Ser. Nos. 60/217,246, titled “Cartridge Loader and Methods”, filed Jul. 10, 2000; 60/364,731, titled “System, Method, and Product for High-Resolution Scanning of Biological Materials”, filed Mar. 15, 2002; 60/396,457, titled “High-Throughput Mircoarray Scanning System and Method”, filed Jul. 17, 2002; and U.S. patent application Ser. No. 09/691,702 titled “Cartridge Loader and Methods”, filed Oct. 17, 2000, each of which is hereby incorporated herein by reference in their entireties for all purposes.

Conveniently, a barcode scanner may be employed to identify the cartridge contents to the host computer. The barcodes may be used as part of a sample tracking systems. In one aspect, a connection may be made to the transport system using a network interface, and a local user interface may be incorporated to facilitate loading and unloading of the cartridges. Further, a non-intrusive alignment mechanism may be used to non-intrusively couple to the scanner. The alignment mechanism may then be used as the sole contact for alignment between the cartridge loader and the scanner. Conveniently, the cartridge loader may be configured to be relatively small in size so as to fit on a bench top and be installable by a single person.

In some embodiments the arrays are washed in an array wash station. Fluidics stations are available from Affymetrix, Inc., Santa Clara, Calif. See U.S. Pat. Nos. 6,114,122, 6,391,623 and 6,422,249 which are incorporated herein by reference.

In some embodiments, the exemplary computer system includes a processor; and a memory being coupled with the processor, the memory storing a plurality of machine instructions that cause the processor to perform the method step of analyzing the hybridization to determine the genotype.

A software system is used to make genotype calls using data generated from Affymetrix GeneChip® mapping arrays, available from Affymetrix, Inc., Santa Clara, Calif. Preferred software is GDAS Software available from Affymetrix, Inc., see U.S. Provisional Patent Application No. 60/423,073 filed Nov. 1, 2002. Software may be implemented in, for example, ANSI standard C code.

GDAS also provides software applications useful for high throughput genotyping. A sequence data manager may manage the functions of analyzing the emission intensity values contained within probe array data files. The data manager may concurrently analyze a plurality of samples that could, for instance, include 40 or more samples.

The data manager may implement genotyping algorithms for the analysis of emission intensity data that, for example, may be derived from probe arrays designed to interrogate DNA sequences. The probe arrays may in some implementations require many copies of a selected DNA sequence in order to obtain reliable data. Many copies of a DNA sequence may be produced by a process such as PCR.

For additional high throughput methods see U.S. patent application Ser. No. 10/028,482 filed Dec. 12, 2001 and PCT application No. US02/41478 each of which is incorporated herein in their entireties by reference. Methods of genotyping using a nucleic acid array following complexity reduction have been described, for example, in U.S. Pat. No. 6,361,947, 6,391,592 and U.S. patent application Ser. Nos. 09/916,135, 09/920,491, 09/910,292, 10/264,945 and 0036069-A1 and in Dong S. et al. (2001), Genome Res., 11:1418-1424, each of which are incorporated herein by reference in their entireties.

### EXAMPLES

### Example 1

Preparation of Genomic DNA. Determine the concentration of the genomic DNA and dilute the working stocks to 50 ng/μL using reduced EDTA TE buffer (0.1 mM EDTA, 10 mM Tris HCl, pH 8.0). For high throughput assays, aliquot 5 μL (50 ng/μL) of each diluted genomic DNA into each well of a 96-well plate. Make multiple replicates of the plates if needed.

Reagent Preparation and Storage. Store the reagents necessary for the restriction digestion, ligation and PCR steps should in the pre-PCR clean room (or area for the DNA template and free of PCR products) to minimize cross contamination between samples. To avoid re-entering the pre-PCR clean room after entering the PCR-Staging Room or the Main Lab, aliquot each of the reagents in the pre-PCR clean room before starting the rest of the experiment.

Restriction enzyme digestion of unamplified genomic DNA samples. In the pre-PCR clean area, prepare the following Digestion Master Mix ON ICE (for multiple samples make a 5% excess):For XbaI mix 10.5 μL H₂O, 2 μL NE buffer 2 (10×), 2 μL BSA (10× (1 mg/mL)), and 0.5 μL Xba I (20 U/μL) for a total of 15 μL For HindIII mix 10.5 μL H₂O2 μL NE buffer 2 (10×) 2 μL BSA (10× (1 mg/mL)) and 0.5 μL Hind III (20 U/μL) for a total of 15 μL. Enzymes, buffers and BSA may be purchased, for example, from New England Biolabs.

In the PCR staging area add 5 μL ILL genomic DNA (50 ng/μL) to each well of 96-well plate. Total amount of genomic DNA is 250 ng for each Restriction Enzyme. Aliquot 15 μL of the Digestion Master Mix to each well of the 96-well plate containing genomic DNA. To expedite the aliquoting, the master mix can be first divided into 8 or 12 microwell strips and then dispensed into the plate with an 8-channel or 12-channel pipette. Pipet up and down for several times to mix the genomic DNA and digestion mix. Cover the plate with a plate cover and seal tightly, vortex, and spin briefly. Place the plate in a thermal cycler and run the following program: 120 minutes 37° C. 20 minutes 70° C. Hold 4° C. store the sample at −20° C. if not proceeding to the next step.

Ligation step. Perform the ligation step in the PCR clean area. Prepare a Ligation Master Mix on ice, for multiple samples make a 5% excess: for XbaI use 1.25 μL Adaptor Xba (5 μM) (available from Affymetrix), 2.5 μL T4 DNA Ligase buffer (10×), 0.625 μL T4 DNA Ligase (400 U/μL), and 0.625 μL H₂O. For HindIII use 1.25 μL Adaptor Hind III (5 μM) (available from Affymetrix), 2.5 μL T4 DNA Ligase buffer (10×), 0.625 μL T4 DNA Ligase (400 U/μL), and 0.625 μL H₂O. Aliquot 5 μL of the Ligation Master Mix into each 20 μL digested DNA sample. To expedite the aliquoting, the Ligation Master Mix can be first divided into 8 or 12 microwell strips and then dispensed into the wells of the plate with an 8-channel or 12-channel pipette. Pipet up and down several times to mix. Cover the plate with plate cover and seal tightly, vortex at medium speed for 2 seconds, and spin briefly. Place the plate in a thermal cycler and run the following program: 120 minutes at 16° C., 20 minutes at 70° C. and hold at 4° C. Store samples at −20° C. if not proceeding to the next step. Dilute each DNA ligation reaction by adding 75 μL of molecular biology-grade water to 25 μL reaction.

PCR. Program the thermal cycler in advance. Make sure the ligated DNA from the ligation step was diluted to 100 μL with water. Prepare PCR Master Mix in Pre-PCR Clean room. Set up PCRs in PCR Staging Area. Prepare 4 PCRs for each sample (4 PCRs are used for each array).

Prepare the following PCR Master Mix on ice for Xba I or Hind III ligation reactions and vortex at medium speed for 2 seconds (for multiple samples make a 5% excess). For each PCR reaction mix 44 μL H₂O, 10 μL Pfx Amplification Buffer (10×), 10 μL PCR Enhancer (10×), 2 μL MgSO₄ (50 mM), 12 μL dNTP (2.5 mM each), 10 μL PCR Primer (10 μM), and 2 μL Platinum Pfx Polymerase (2.5 U/μL), Invitrogen. Transfer 10 μL of each of the diluted ligated DNA from the 96-well plate into the corresponding wells of four new PCR plates using an 8- or 12-channel pipette. Add 90 μL PCR master mix to obtain a total volume of 100 μL. It is convenient to dispense the PCR Master Mix with a repetitive dispenser (such as Gilson Distriman®, available from Ranin) or pipet the PCR Master Mix from a solution basin (Labcor Products, Inc., Cat. No. 730-014; available from PGC Scientifics) with an 8-channel or 12-channel pipette.

In the PCR staging area add 90 μL PCR Master Mix to 10 μL Diluted ligated DNA (from Ligation step). Seal the plate with plate cover, vortex at medium speed for 2 seconds, and spin briefly. Four PCR reactions may be used to produce sufficient product for hybridization to one array (each reaction=100 μL). In the main lab run the following PCR protocols on either an MJ DNA Engine Tetrad or GeneAmp PCR System 9700. PCR protocols for MJ and PE thermal cyclers are different as listed below. MJ DNA Engine Program: use Heated Lid and Calculated Temperature. Program the thermal cycler in advance with the following protocol: 94° C. for 3 minutes, 30 cycles of 15 seconds at 94° C., 30 seconds at 60° C. and 60 seconds at 68° C., finally 7 minutes at 68° C. and hold at 4° C.

If using the Gene Amp PCR System 9700 use the following program: specify 100 μL volume and maximum mode, 1 cycle of 3 minutes at 94° C., 30 cycles of 30 seconds at 94° C., 45 seconds at 60° C. and 60 seconds at 68° C., 1 cycle of 7 minutes at 68° C. then hold at 4° C.

PCR Purification and Elution with QIAGEN MinElute 96 UF PCR Purification Plate. Connect a vacuum manifold to a suitable vacuum source able to maintain ˜800 mbar, e.g., QIAvac 96 or QIAvac Multiwell Unit (QIAGEN). Place a waste tray inside the base of the manifold. Place a MinElute 96 UF PCR Purification Plate on top of the manifold. Cover wells that are not needed with PCR plate cover. Turn on the vacuum. To cover the unneeded wells, a PCR plate cover can be placed on top of the MinElute plate. Apply pressure to make the cover stick to the plate. Then using a razor, cut between the needed and unneeded wells. Remove the portion that covers the needed wells.

Pipet the PCR samples onto the MinElute plate. For PCR samples prepared in four 96-well PCR plates, an 8- or 12-channel pipette can be used to transfer each row of 12 samples in the PCR plates to the corresponding row of the MinElute plate. With the vacuum on, the four PCR reactions for each sample (400 μL) can be combined into one well of the MinElute plate. Maintain the ˜800 mbar vacuum until the wells are completely dry. Wash the PCR products by adding 50 μL molecular biology water and dry the wells completely (taking about 20 minutes). Repeat this step 3 times. Switch off vacuum source and release the vacuum. Carefully remove the MinElute plate from the vacuum manifold. Gently tap the MinElute plate on a stack of clean absorbent paper to remove any liquid that might remain on the bottom of the plate. Add 40 μL EB buffer to each well. Cover the plate with PCR plate cover film. Moderately shake the MinElute plate on a plate shaker, e.g., Jitterbug (Boekel Scientific, model 130000), for 5 minutes. Recover the purified PCR product by pipetting the eluate out of each well. For easier recovery of the eluates, the plate can be held at a slight angle.

Quantification of Purified PCR Product. Use spectrophotometric analysis to determine the purified PCR product yield. If available, a plate reader is used for efficient DNA concentration determination. Add 4 μL of purified PCR product to 156 μL molecular water (40-fold dilution) and mix well. Read the absorbance at 260 nm. Ensure that the reading is in the quantitative range of the instrument (generally 0.2 to 0.8 OD). Apply the convention that 1 absorbance unit at 260 nm equals 50 μg/mL for double-stranded PCR product. Normalize the DNA concentration to 40 μg of PCR product per 45 μL solution by adding EB buffer (10 mM Tris-HCl, pH 8.5). Transfer 45 μL (40 μg) of each of the purified DNA to corresponding wells of a new plate for fragmentation. If 40 μg of PCR product has a volume less than 45 μL, make up the volume by adding EB Buffer (10 mM Tris-Cl, pH 8.5).

Fragmentation. In the Main Lab pre-heat a thermal cycler to 37° C. Add 5 μL 10×Fragmentation Buffer to 45 μL Purified PCR product (40 μg in EB buffer) on ice and vortex at medium speed for 2 seconds. Dilute GeneChip Fragmentation Reagent using the following formula Y=(0.04 U/μL×150 μL)/X U/μL where Y=number of μL of stock Fragmentation Reagent, X=number of units of stock Fragmentation Reagent per μL (see label on tube), 0.04 U/μL=final concentration of diluted Fragmentation Reagent, 150 μL=final volume of diluted Fragmentation Reagent, this is enough for 20 reactions. As the concentration of stock Fragmentation Reagent (U/μL) may vary from lot to lot, the concentration may be checked before conducting the dilution.

Dilute the stock of Fragmentation Reagent to 0.04 U/μL using Fragmentation Buffer and Molecular Biology Water on ice and vortex at medium speed for 2 seconds. For example, if the fragmentation reagent is 2 units/μL use 2 μL Fragmentation Reagent, 15 μL 10×Fragmentation Buffer, 132 μL H₂O or if the Fragmentation Reagent is 3 units/μL use 2 μL Fragmentation Reagent, 15 μL Fragmentation Buffer, and 133 μL H₂O. Divide the diluted Fragmentation Reagent into 8 or 12 microwell strips on ice. Add 5 μL of diluted Fragmentation Reagent (0.04 U/μL) with an 8- or 12-channel pipette to the fragmentation plate containing Fragmentation Mix on ice. Pipet up and down several times to mix. The total volume for each sample is listed below. For 40 μg of purified PCR product, a total of 0.2 U of Fragmentation Reagent is used in a final reaction volume of 55 μL. Cover the fragmentation plate with a plate cover and seal tightly. Vortex the fragmentation plate at medium speed for 2 seconds, and spin briefly.

Place the fragmentation plate in pre-heated thermal cycler (37° C.) as quickly as possible and run the following program: 35 minutes at 37° C., 15 minutes at 95° C. and hold at 4° C. Spin the plate briefly after fragmentation reaction. Dilute 4 ILL of fragmented PCR product with 4 μL gel loading dye and run on 4% TBE gel at 120V for 30 minutes to 1 hour. Proceed immediately to the labeling step.

Labeling. Prepare Labeling Mix as master mix on ice in the main lab and vortex at medium speed for 2 seconds (for multiple samples make a 5% excess) using reagents available from Affymetrix. Labeling Mix is made by mixing 14 μL5×TdT Buffer, 2 μL GeneChip DNA Labeling Reagent (7.5 mM) and 3.5 μL TdT (30 U/μL). Aliquot 19.5 μL of Labeling Master Mix into the fragmentation plate containing 50.5 μL of fragmented DNA samples as follows (To expedite the aliquoting, the Labeling Master Mix can be first divided into 8 or 12 microwell strips and then dispensed into the wells of the plate with an 8- or 12-channel pipette. Pipet up and down several times to mix.). Seal the plate tightly with a plate cover. Vortex the plate at medium speed for 2 seconds, and briefly spin the plate. The plates/reaction tubes should be securely sealed prior to running the program in order to minimize solution loss due to evaporation. Run the following program: 37° C. for 2 hours, 95° C. for 15 minutes and hold at 4° C. Briefly spin the plate after the labeling reaction. Samples can be stored at −20° C. if not proceeding to next step.

Target Hybridization. Prepare MES stock. For 1000 mL 12×MES Stock (1.22 M MES, 0.89 M [Na⁺]) mix 70.4 g MES-Free Acid monohydrate, 193.3 g MES Sodium Salt, 800 mL Molecular Biology Grade water, mix and adjust volume to 1,000 mL, the pH should be between 6.5 and 6.7. Filter through a 0.2 μm filter. Store MES stock at between 2° C. and 8° C., and shield from light. Discard solution if it turns yellow.

Prepare the following Hybridization Cocktail Master Mix (for multiple samples make a 5% excess): 12 μL MES (12×; 1.22 M)), 13 μL DMSO (100%), 13 μL Denhardt's Solution (50×), 3 μL EDTA (0.5 M), 3 μL Herring Sperm DNA (10 mg/mL), 2 μL Oligonucleotide Control, 3 μL Human Cot-1 (1 mg/mL), 1 μL Tween-20 (3%) and 140 μL TMACL (5M). Transfer each of the labeled samples from the plate to a 1.5 mL Eppendorf tube. Aliquot 190 μL of the Hybridization Cocktail Master Mix into the 70 μL of labeled DNA samples (this mix can be stored at −20° C. before proceeding to the next step. Heat the 260 μL of hybridization mix and labeled DNA at 95° C. in a heat block for 10 minutes to denature. Cool down on crushed ice for 10 seconds. In a preferred embodiment do not leave on ice for longer than 10 seconds. Spin briefly in a microfuge to collect any condensate. If there is anything that has come out of the solution, pipette briefly to resuspend before adding solution to the array. Place the tubes at 48° C. for 2 minutes. Inject 200 μL denatured hybridization into the array. Hybridize at 48° C. for 16 to 18 hours at 60 rpm.

Mapping Arrays: Washing, Staining, and Scanning. The Fluidics Station 400 and 450/250 were used to automate the washing and staining of GeneChip® mapping probe arrays, and the probe arrays were scanned using the GeneChip® Scanner 3000. Prepare wash buffers. Wash A (non-stringent wash buffer) is 6×SSPE, 0.01% Tween 20. For 1000 mL mix 300 mL of 20×SSPE, 1.0 mL of 10% Tween-20, and 699 mL of water. Filter through a 0.2 μm filter and store at room temperature. Wash B: Stringent Wash Buffer is 0.6×SSPE, 0.01% Tween 20. For 1000 mL mix 30 mL of 20×SSPE, 1.0 mL of 10% Tween-20 and 969 mL of water. Filter through a 0.2 μm filter and store at room temperature. Prepare 1 mg/mL Streptavdin Stock by resuspending 1 mg in 1 mL Molecular Biology Grade water and store at 4° C. Prepare 0.5 mg/mL Anti-Streptavdin Antibody by resuspending 0.5 mg in 1 mL of water. Store at 4° C.

Wash and stain the array according to the instructions provided in the GeneChip Mapping Array manual. After 16 hours of hybridization, remove the hybridization cocktail from the probe array and set it aside in a microcentrifuge vial. Store on ice during the procedure or at −80° C. for long-term storage. Fill the probe array completely with 250 μL of Non-Stringent Wash Buffer. Prepare streptavidin solution mix by mixing 495 μL Stain Buffer and 5.0 μL 1 mg/mL Streptavidin. Prepare antibody stain solution by mixing 495 μL Stain Buffer and 5 μL 0.5 mg/mL biotinylated antibody. Prepare SAPE Stain Solution by mixing 495 μL Stain Buffer and 5.0 μL1 mg/mL Streptavidin Phycoerythrin (SAPE).

### Example 2

High throughput preparation of reduced complexity samples. To increase sample throughputs, procedures were carried out in 96-well plates. For each individual assayed, 250 ng of genomic DNA was digested with 10 U of Xba I (New England BioLabs) in a volume of 15 μL for 2 hours at 37° C. Following heat inactivation at 70° C. for 20 minutes, 0.25 μM of adaptor (5′phosphate-CTA GAG ATC AGG CGT CTG TCG TGC TCA TAA-3′ (SEQ ID NO 1), and 5′-ATT ATG AGC ACG ACA GAC GCC TGA TCT-3′ (SEQ ID NO 2)) was ligated to the digested DNA with T4 DNA Ligase (New England BioLabs) in 25 μL for 2 hours at 16° C. The ligation was stopped by heating to 70° C. for 20 minutes, and then diluted 4-fold with water. For each sample, four PCRs were run using 10 μL of the diluted ligation reaction (25 ng of starting DNA) in 100 μL volumes containing 0.75 μM of primer (sense strand of adaptor), 0.25 mM dNTPs, 2.5 mM MgCl₂, 10 U AmpliTaq Gold® (Applied Biosystems), and PCR Buffer (Applied Biosystems). 35 cycles of PCRs were done in either MJ DNA Engine Tetrad (MJ Research) or GeneAmp PCR System 9700 (Applied Biosystems) cyclers. The cycling program in the MJ Tetrads was 95° C. denaturation for 20 seconds, 59° C. annealing for 15 seconds, and 72° C. extension for 15 seconds. The denaturation, annealing and extension times were each increased to 30 seconds when using the GeneAmp cycler. As a check, 3 μL of PCR products were visualized on 2% TBE agarose gels to confirm the size range of amplicons. PCR products from the four reactions were combined and purified over MinElute 96 UF PCR Purification plates (Qiagen). PCR amplicons from the four 100 μL reactions were recovered in 40 μL of EB buffer (Qiagen). PCR yields, based on absorbance readings at 260 nm, were typically ˜30 μg. To allow efficient hybridization to the 25-mer oligonucleotides on the array, PCR amplicons were fragmented with DNAse I (Amersham Biosciences). 0.24 U of DNAse I was added to 20 ug of purified PCR amplicons in a 55 μL volume containing 50 mM potassium acetate, 20 mM Tris-acetate, 10 mM magnesium acetate, and 1 mM dithiothreitol for 30 minutes at 37° C., followed by heat inactivation at 95° C. for 15 minutes. Fragmentation products were visualized on 4% TBE agarose gels. The 3′ ends of the fragmented amplicons were biotinlyated by adding 143 μM of a proprietary DNA labeling reagent (Affymetrix) using Terminal Deoxynucleotidyl Transferase (Promega) in a 70 μL volume containing 100 mM cacodylic acid (pH 6.8), 0.1 mM dithiothreitol, and 1 mM CoCl₂ for 2 hours at 37 ° C., followed by heat inactivation at 95° C. for 15 minutes.

### Example 3

Workflow for GeneChip High Throughput 10K Mapping Assay. Phase 1 is sample preparation for 1400 individual samples to be performed in 10 days. The lab setup is as follows: 8 96 well heating blocks capable of thermal cycling placed in low template room, 12 96 well PCR modules, 8 Qiagen QiaVac and MinElute plates for purification, a plate reader for DNA quantification, one 12 channel pipettes and two repeating pipettes.

During week 1 samples are prepared for 768 samples. Start with 50 ng/μl genomic DNA working stocks in 8 96 well plates. The genomic DNAs are in water. Week 2 is used to prepare samples from an additional 768 samples using the protocol for week 1. During weeks 3 and 4 each sample is hybridized to one copy of a genotyping array. Table 1 shows a workflow for a high throughput 10K Mapping Assay. FTE is full time employee and is an indication of how many employee resource hours are used.

TABLE 1

| Day 1 | Restriction enzyme digestion and ligation |
|---|---|
| 9:00 am | Thaw 8 plates of genomic DNA stock solutions, 100x BSA and NEB2 |
| | buffer.(15 min) |
| 9:15 | Prepare Xba I digestion master mix, preparing 10% extra |
| 9:25 | Aliquot 126 ul of the master mix into each tube of 8 by 12 tube strips with a |
| | repeating pipette |
| 9:30 | Aliquot 15 ul of digestion mix into each well of the 8 96 well plates with 12 |
| | channel pipette. Pipette up and down. Seal with cover films. |
| 10:15 | Vortex and spin down |
| 10:30 | Digestion with a thermal cycler for 90 minutes, LUNCH BREAK |
| 12:30 | Thaw adaptor Xba and T4 DNA ligase buffer |
| 12:45 | Prepare ligation master mix (10% excess) |
| 12:55 | Aliquot 126 ul of the ligation master mix (enough for 4 plates) into each tube of |
| | two 12-tube strips using a repeating pipette. |
| 1:00 | Aliquot 3.75 ul of the ligation master mix from the strips into each well of the 8 |
| | plates using a 12 channel pipette. |
| 1:45 | Aliquot 84 μl T4 DNA ligase into each tube of a 12 tube strip |
| 1:50 | Aliquot 1.25 μl T4 DNA ligase into each well of the 8 plates with a 12 channel |
| | pipette |
| 2:25 | Vortex and spin down |
| 2:35 | Ligation on thermal cyclers for 150 minutes |
| 5:05 | Ligation finished, store at 4° C. |
| Day 2 | PCR for two FTEs |
| 9:00 am | Take out PCR reagents. While waiting for them to be thawed, aliquot 2.5 μl of |
| | each ligated DNA from 4 plates of ligated DNA into the corresponding wells of |
| | 16 new 96-well plates with a 12-channel pipette. |
| 9:45 | Prepare PCR master mix |
| 10:15 | Prepare and label 16 PCR plates |
| 10:45 | Add PCR master mix to each well of the 16 PCR plates |
| 11:45 | Vortex and spin down |
| 12:00 | Run PCR for the first 16 plates (150 minutes) |
| 12:30 | Aliquot 2.5 ul of each ligated DNA from another 4 plates of ligated DNA into |
| | the corresponding wells of 16 new 96-well plates with a 12-channel pipette. |
| 1:15 | Add PCR Master Mix to each well of the 16 PCR plates using repeating |
| | pipettes. |
| 2:15 | Vortex and spin down |
| 2:30 | Run PCR for the 2^{nd} set of 16 plates |
| 5:00 | PCR complete store at 4° C. |
| DAY 3 | PCR purification and DNA quantification for 8 plates of samples-2 FTEs |
| 9:00 am | Run 2% agarose gels to check PCR and take photos |
| 11:00 | Set up vacuum and MinElute plates |
| 11:10 | Transfer 4 plates of PCR products (for the same plate of individuals) to one |
| | MinElute plate with a 12-chennel pipette.(15 min/plate; total time: 60 min) |
| 12:10 | Finish loading PCR samples to MinElute plates. (Lunch break while PCR |
| | products are being filteres) |
| 1:00 | Wash the first dried MinElute plate with water by transferring 50 μl water from |
| | a solution basin to the wells with a 12 channel pipette. Wash the remaining 3 |
| | plates when dried. |
| 2:30 | Add 50 μl EB buffer to each well and shake for 2 min |
| 2:50 | Recover the dissolved DNA from the MinElute plates |
| 3:30 | Pipette 4 μl solution from the MinElute wells to 96 well plate for plate reader |
| | and mix with 156 μl water |
| 4:10 | DNA quantification with a plate reader |
| 4:40 | Transfer concentration data and calculate volumes of DNA solution and EB |
| | buffer needed to obtain 20 μg DNA in a volume of 45 μl. |
| 5:10 | Store DNA at 4° C. |
| Day 4 | Set up for fragmentation-1 FTE |
| 9:00 am | Mix DNA and EB to obtain 20 μg/45 μl DNA for plate 1 |
| 10:30 | Mix DNA and EB to obtain 20 μg/45 μl DNA for plate 2 |
| 12:00 | Lunch break |
| 12:30 | Mix DNA and EB to obtain 20 μg/45 μl DNA for plate 3 |
| 2:00 | Mix DNA and EB to obtain 20 μg/45 μl DNA for plate 4 |
| 3:30 | Thaw fragmentation buffer |
| 3:45 | Aliquot 22 μl of fragmentation buffer into each wells of a 12-tube strip. |
| 4:00 | Mix 5 ul of fragmentation buffer with the 45 ul DNA solution with a 12 |
| | channel pipette. Pipette up and down to mix |
| 5:00 | Store solution at 4° C. |
| Day 5 | Fragmentation and labeling-one FTE |
| 9:00 am | Thaw 10X fragmentation buffer. Start the fragmentation program and |
| | equilibrate the heating block to 37° C. |
| 9:15 | Dilute fragmentation reagent stock to 0.24 U/μl by mixing the following |
| | reagents in the following order: 468 μl water, 60 μl 10X fragmentation buffer, |
| | and 72 μl 2 U/μl fragmentation reagent. |
| 9:25 | Aliquot 48 μl of diluted fragmentation reagent into each tube of a 12-tube strip. |
| 9:27 | Mix 5 μl diluted fragmentation reagent with each DNA sample with a 12 |
| | channel pipette. Pipette up and down quickly several times. |
| 9:29 | Vortex and spin down for 1 minute |
| 9:32 | Start fragmentation |
| 9:35 | Fragmentation for second plate |
| 10:10 | Fragmentation for third plate |
| 10:45 | Fragmentation for fourth plate |
| 11:30 | Finish fragmentation. Set up for 4% agarose gel. |
| 12:00 | Run gel for 45 minutes. Lunch break |
| 12:45 | Take out and thaw labeling buffer and DNA labeling reagent. Take pictures of |
| | the gels. |
| 1:00 | Start labeling program and equilibrate the heating block to 37 C. Prepare |
| | labeling master mix for 4 plates of samples. |
| 1:15 | Aliquot 163 μl labeling master mix into each tube of 4 12 tube strips using a |
| | repeating pipette |
| 1:25 | Mix 19.4 μl labeling master mix with each DNA sample with a 12 channel |
| | pipette. Pipette up and down to mix |
| 2:10 | Vortex and spin down |
| 2:15 | Run labeling protocols (135 minutes) |
| 4:30 | Finish labeling. Store samples at −20° C. |
| Week 2 | Finish the sample preparation for the rest of the samples. |

### Example 4

Cleanup of DNA suspected of being contaminated with inhibitors. If a genomic DNA sample is suspected of containing one or more inhibitors that result in poor amplification of the sample during WGSA treatment it may be desirable to cleanup the DNA prior to the WGSA treatment. The following method may be used. Add 0.5 volumes of 7.5 M NH₄OAc, 2.5 volumes of absolute ethanol (stored at −20° C.), and 0.5 μl of glycogen (5 mg/mnL) to 250 ng genomic DNA. Vortex and incubate at −20° C. for 1 hour. Centrifuge at 12,000×g in a microcentrifuge at room temperature for 20 minutes. Remove supernatant and wash pellet with 0.5 mL of 80% ethanol. Centrifuge at 12,000×g at room temperature for 5 minutes. Remove the 80% ethanol and repeat the 80% ethanol wash one more time. Re-suspend the pellet in TE (10 mM, pH 8.0, 0.1 mM EDTA, pH 8.0). This cleanup method may be used, for example, when samples are obtained from formalin fixed paraffin embedded tissue samples.

### Conclusion

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

1. A system for genotyping a plurality of single nucleotide polymorphisms (SNPs) in a plurality of genomic DNA samples comprising:
a sample preparation method comprising a non locus-specific amplification step to amplify genomic DNA, a fragmentation step to fragment amplified genomic DNA; and a labeling step that generates labeled amplified fragments;
a sample preparation kit comprising at least two containers, wherein the first container contains one or more amplification reagent stocks for the amplification step and the second container contains one or more fragmentation reagent stocks for the fragmentation step and one or more labeling reagent stocks for the labeling step;
a first low copy lab area wherein the amplification reagent stocks of the first container are stored and wherein an amplification reagent master mix is assembled;
a second low copy area where the plurality of genomic DNA samples is stored and wherein a plurality of amplification reactions is assembled, wherein each amplification reaction comprises an aliquot of the amplification reagent master mix and an aliquot of each of said plurality of genomic DNA samples;
a high copy lab area wherein said amplification, fragmentation and labeling steps are performed to generate labeled, amplified fragments from each of said plurality of genomic DNA samples;
instructions to lab personnel to restrict movement of amplified genomic DNA samples, lab personnel and equipment from the high copy lab area to said first and second low copy lab areas;
a plurality of genotyping arrays wherein each array in the plurality comprises a set of at least 200,000 probes comprising at least 10,000 probe sets wherein a probe set comprises probes that are complementary to a first allele of a SNP and probes that are perfectly complementary to a second allele of the SNP;
a method for hybridizing said labeled, amplified fragments to said plurality of genotyping arrays to generate a hybridization pattern for each genomic DNA sample; and,
a computer system for analyzing each hybridization pattern to determine the genotype of a plurality of SNPs.

2. The system of claim 1 wherein the non locus specific primers are selected from the group consisting of a universal primer, random primers and degenerate primers.

3. The system of claim 1 wherein the sample preparation kit comprises a third container containing a reference genomic DNA sample.

4. The system of claim 1 wherein the amplification reagent stocks in said first container comprise a DNA polymerase, dNTPs, and a concentrated buffer solution.

5. The system of claim 1 wherein the fragmentation reagent stocks comprise DNase I and a concentrated buffer solution.

6. The system of claim 1 wherein the labeling reagent stocks comprise a terminal deoxynucleotidyl transferase, a concentrated buffer solution and a biotinylated nucleotide.

7. The system of claim 1 further comprising a robotic device for handling multiwell plates.

8. The system of claim 1 wherein said computer system comprises a processor; and a memory being coupled with the processor, the memory storing a plurality of machine instructions that cause the processor to perform the method step of analyzing the hybridization to determine the genotype and wherein said system further comprises a sample tracking system wherein the sample tracking system is selected from the group consisting of a bar code system and an electromagnetic encoding system.

9. The system of claim 1 wherein the second low copy lab area comprises a first thermal cycler and the high copy lab area comprises a second thermal cycler.

10. A method of determining the genotype of a plurality of SNPs in a genomic DNA sample comprising:
storing a plurality of amplification reagent stocks in a first low copy lab area wherein genomic DNA samples and amplified genomic DNA samples are not intentionally brought into said first low copy lab area;
assembling an amplification reagent master mix in said first low copy lab area, wherein said amplification reagent master mix comprises aliquots of amplification reagent stocks;
transporting said amplification reagent master mix to a second low copy lab area wherein unamplified genomic DNA samples are stored;
assembling an amplification reaction in said second low copy lab area, wherein the amplification reaction comprises an aliquot of unamplified genomic DNA and an aliquot of the amplification reagent master mix;
transporting said amplification reaction to a high copy area, wherein reagent stocks for fragmentation and labeling of amplified samples are stored;
incubating said amplification reaction under amplification conditions in said high copy area;
fragmenting said amplification reaction in said high copy area to generate fragments;
labeling the fragments in the high copy area;
hybridizing the labeled fragments to a genotyping array and analyzing the hybridization pattern to determining the genotype of a plurality of SNPs.

11. The method of claim 10 wherein the amplification reagent master mix comprises an aliquot of DNA polymerase, dNTPs and concentrated buffer solution.

12. The method of claim 10 wherein said genotyping array comprises at least 200,000 different probes comprising at least 10,000 probe sets wherein a probe set comprises at least 20 probes that are each complementary to a 20 to 30 base region comprising a human single nucleotide polymorphism and wherein the probe set comprises probes that are perfectly complementary to a first allele of the SNP and probes that are perfectly complementary to a second allele of the SNP.

13. The method of claim 10 further comprising:
storing a plurality of ligation reagent stocks in said first low copy lab area; and,
assembling a ligation reagent master mix in said first low copy lab area, wherein said ligation reagent master mix comprises aliquots of said plurality of ligation reagent stocks.

14. The method of claim 13 wherein said plurality of ligation reagent stocks comprise ligation buffer, a DNA ligase and an adaptor.

15. A kit for amplifying a genomic DNA sample comprising:
a first container containing an adaptor and an amplification primer and optionally comprising reagents selected from the group consisting of a ligase, a ligase buffer, a DNA polymerase, and a buffer for the DNA polymerase;
a second container containing a reference genomic DNA sample;
a third container containing a DNase, a DNase buffer, a terminal deoxynucleotidyl transferase, a terminal deoxynucleotidyl transferase buffer, and a labeled nucleotide; and
instructions for storing the contents of the first container in a first low copy lab area, the contents of the second container in a second low copy lab area and the contents of the third container in a high copy lab area.

16. The kit of claim 15 wherein said amplification primer is selected from the group consisting of a universal primer, random primers and degenerate primers.

17. The kit of claim 15 wherein said instructions for storing are provided as a first label affixed to said first container, a second label affixed to said second container and a third label affixed to said third container wherein said first label comprises instructions for storage of said first container or the contents thereof, said second label comprises instructions for storage of said second container or the contents thereof, and said third label comprises instructions for storage of said third container or the contents thereof.

18. A method for genotyping a panel of more than 10,000 SNPs in at least 96 individuals comprising:
isolating a genomic DNA sample from each of the at least 96 individuals;
fragmenting an aliquot of each genomic DNA sample in a first fragmentation step;
ligating an adaptor to the fragments in each fragmented genomic DNA sample to generate an adaptor-ligated genomic DNA sample for each individual, wherein the first fragmentation and adaptor ligation steps are performed in a first low copy lab area;
assembling an amplification reaction for each individual comprising an aliquot of adaptor ligated genomic DNA and reagents for amplification wherein the amplification reactions are assembled in said low copy lab area;
incubating the amplification reactions under amplification conditions to generate amplicons in a high copy lab area, wherein amplified samples from the high copy lab area are not transported back to the first low copy lab area after amplification;
fragmenting the amplicons to generate fragmented amplicons in a second fragmentation step;
end labeling the fragmented amplicons with a detectable label;
hybridizing the end labeled, fragmented amplicons from each genomic DNA sample to a genotyping array to generate a hybridization pattern for each genomic DNA sample; and,
analyzing the hybridization pattern for each genomic DNA sample with a computer system to determine the genotype of a plurality of SNPs in each of the at least 96 individuals.

19. The method of claim 18 wherein the genotyping array comprises allele specific probes for each allele of at least 10,000 human SNPs.

20. The method of claim 18 wherein the genotyping array comprises allele specific probes for each allele of at least 100,000 human SNPs.

21. The method of claim 18 wherein the genotyping array comprises allele specific probes for each allele of at least 10,000 mouse or rat SNPs.

22. The method of claim 18 wherein reagent stocks for the first fragmentation, the adaptor ligation and the amplification are stored in a second low copy lab area and reagent master mixes for the first fragmentation, adaptor ligation and amplification steps are assembled in said second low copy lab area.


SEQUENCE LISTING (2 sequences)

SEQ ID NO: 1 - DNA, 30 bp, artificial sequence
  synthetic oligonucleotide
ctagagatcaggcgtctgtcgtgctcataa

SEQ ID NO: 2 - DNA, 27 bp, artificial sequence
  synthetic oligonucleotide
attatgagcacgacagacgcctgatct